(19) United States
(12) Patent Application Publication
(10) Pub. No.: US 2017/0035928 A1
(43) Pub. Date: Feb. 9, 2017

(54) Congruent Opposing Action Wound Dressing

(71) Applicant: Joshua D. Smith, Nashville, TN (US)

(72) Inventor: Joshua D. Smith, Nashville, TN (US)

(21) Appl. No.: 14/564,094

(22) Filed: Dec. 9, 2014

(51) Int. Cl. A61L 15/42; A61F 13/00; A61K 35/741; A61K 9/00; A61L 15/40; A61L 15/44

(52) U.S. Cl. CPC A61L 15/42; A61L 15/40; A61L 15/44; A61L 15/425; A61K 35/741; A61K 9/0014; A61F 13/00063; A61L 2300/40

(57) ABSTRACT

A treatment of a wound involving the topical application of probiotics defined as beneficial microorganisms or cellular nutrients in cooperation with a hydrophobic wound dressing designed to bind microorganisms.

### CROSS REFERENCE TO RELATED APPLICATIONS

This application claims priority of U.S. co-pending Provisional Application No. 61/914,226 filed Dec. 10, 2013, entitled “HYDROPHOBIC WOUND DRESSING WITH MICROBIAL ADDITIVES” the entirety of which is incorporated by reference herein.

### BACKGROUND OF THE INVENTION

Presently hydrophobic wound dressings designed to bind microorganisms preferentially target pathogenic microorganisms by way of hydrophobic interaction which is a method of exploiting the increased cell surface hydrophobicity of pathogenic microorganisms. Even though this method is effective at neutralizing infection causing microorganisms it has little effect on the normal flora within the wound. The benefit of this type of wound dressing is that it aims to prevent or treat infection without damaging healthy cells or normal wound flora as opposed to silver products which kill microbes and damage cells indiscriminately. The importance of creating products which support normal flora can best be illustrated by the NIH funded Human Microbiome Project or by recognizing the beneficial role probiotics play in digestive health.

Though current hydrophobic wound dressings neutralize pathogenic microorganisms, they do nothing to introduce beneficial additives which are designed to support or replace the existing normal flora in the wound.

The present invention significantly enhances the current hydrophobic binding wound dressing technology by combining the topical addition of nutrients identified to stimulate normal flora and cells metabolisms and their reproduction or by adding beneficial microorganisms themselves to the wound environment. Nutrients and beneficial microorganisms may be added together or independently in combination with a hydrophobic wound dressing designed to bind microorganisms.

Beneficial nutrients and microorganisms may be added to a suspension of hydrogel, or similar as the preferred delivery method in combination with a hydrophobic wound dressing designed to bind microorganisms. Alternately these same beneficial additives may be delivered to the wound in a dry form which may be more appropriate for other combinations with collagens or the like.

### BRIEF SUMMARY OF THE INVENTION

The present invention combines existing technology designed to bind microorganisms in the form of a hydrophobic wound dressing thereby targeting pathogenic microorganisms by exploiting the pathogens' higher cell surface hydrophobicity with the simultaneous introduction of hydrophilic beneficial microorganisms and cellular nutrients preferably in the form of a hydrogel suspension acting as the delivery mechanism.

It is therefore shown that the novelty of the present invention is a hydrophobic wound dressing as described in U.S. Pat. No. 4,617,326 and U.S. Pat. No. 7,576,256 combined with beneficial microorganisms and cellular nutrients preferably in the form of a hydrogel suspension. These are referred to as microbial additives.

Since microorganisms are extremely diverse with many classifications, subspecies, and strains; for the purposes of the present invention we will define beneficial microorganisms as microorganisms exhibiting a low cell-surface hydrophobicity or microorganisms that are hydrophilic in nature and therefore not affected by the presence of a hydrophobic wound dressing.

Examples of beneficial nutrients include but are not limited to substances derived from the fermentation process, amino acids, fatty acids, glucose, cellulose, creatine, and the like, or any nutrient which may be broken down and metabolized by beneficial microorganisms encouraging microbial health, growth, and reproduction. These nutrients may also be beneficial to human cells.

It is well known that a hydrophobic wound dressing designed to bind microorganisms is constructed of a hydrophilic substrate coated in a substance to then render the substrate hydrophobic. This can be described as a microbe binding substrate designed to target the binding of pathogens a described in U.S. Pat. No. 4,617,326 and U.S. Pat. No. 7,576,256

To those skilled in the art it will be apparent that the spirit of the invention is one designed to neutralize infection causing microorganisms without harming normal flora or human cells in a wound while topically introducing additives to support beneficial microbial colonies or human cells and that the invention shall not be limited except for the cooperation of these two actions defined as neutralizing pathogens while supporting beneficial flora and human cells with topical additives within a wound.

### BRIEF DESCRIPTION OF SEVERAL VIEWS OF THE DRAWINGS

FIG. 1 shows the wound 1 containing on the surface pathogenic microorganisms 6 and then a microbe binding substrate 3 delivering nutrients 5 and beneficial microbes 4 to the wound.

FIG. 2 shows the present invention 2 making contact with the wound 1 to deliver said nutrients 5 and beneficial microbes 4 while binding pathogenic microorganisms 6 to the microbe binding substrate 3.

FIG. 3 demonstrates the outcome of the present invention 2 and shows the removal of the microbe binding substrate 3 from the wound 1 wherein the nutrients 5 and beneficial microbes 4 have been delivered to the wound 1 and the pathogenic microorganisms 6 have bound to the microbe binding substrate 3.

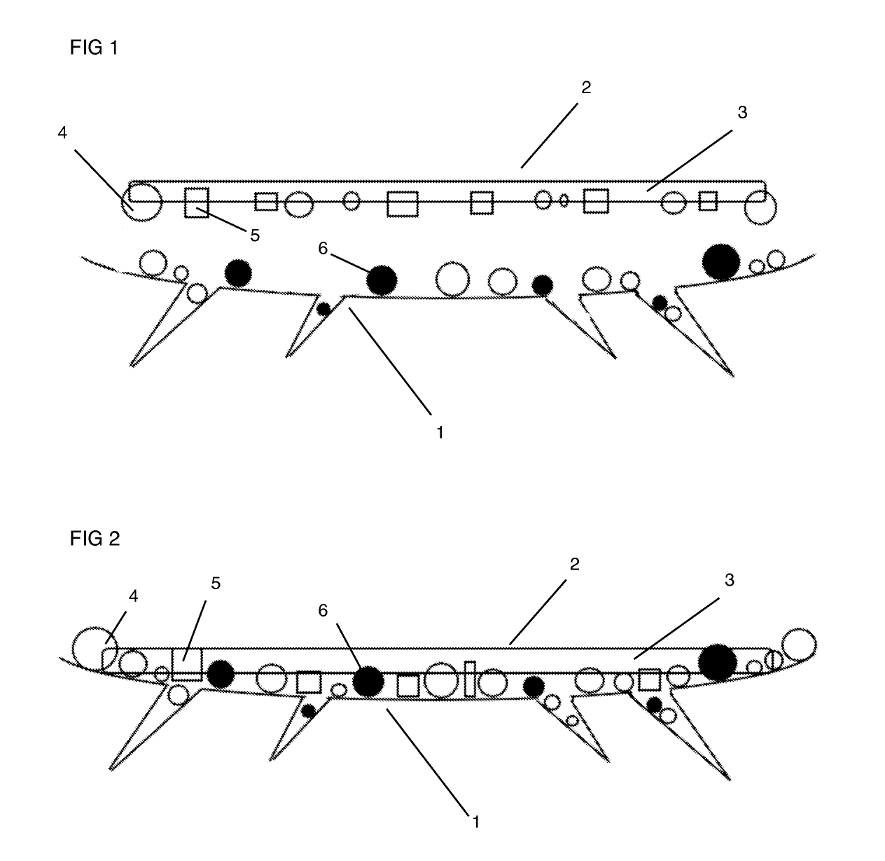

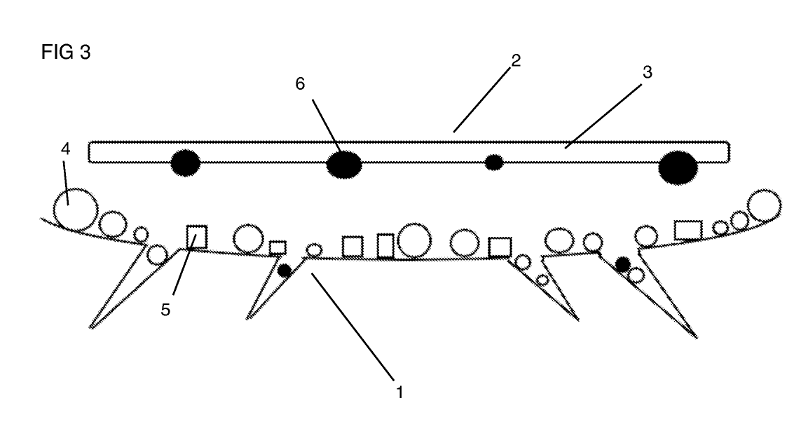

### DETAILED DESCRIPTION OF THE INVENTION

The present invention is ideal for preferentially targeting the treatment of pathogenic microorganisms without harming normal flora or human cells and then topically applying additives which support beneficial microbial colonies and human cells. In cooperation with a microbe binding substrate, topical additives may be either live microbial cultures, nutrients which support beneficial microbes and human cells, or any combination thereof. The preferred delivery method of said topical additives is a hydrogel suspension or the like.

As described in U.S. Pat. No. 4,617,326 and U.S. Pat. No. 7,576,256: the microbe binding substrate may consist of folded acetate gauze and cotton gauze treated with the fatty acid ester DACC (dialkyl carbamoyl chloride) or dioctadecyl-carbamoyl chloride or an alkyl ketene dimer (AKD) so that the fibers have a strong hydrophobic property which cause pathogenic microorganisms in fluids to adhere to the substrate through hydrophobic interaction. The substrate can optionally be rendered cation active. Such substrates have a primary component which has one or more liquid permeable layers of a hydrophobic and possible cationic, bacteria adsorbing, physiologically innocuous material containing a woven or non-woven hydrophilic fabric. The fabric has been rendered hydrophobic by chemical treatment with a compound containing hydrophobic groups. One skilled in the art will understand that future embodiments of the invention may be altered to include other hydrophilic materials, hydrophobic fibers, non-woven substrates, and other materials such as foam and silicone combined with a chemical treatment to render said substrate hydrophobic or enable said substrate to bind microorganisms and toxins by adhesion.

In its basic embodiment the substrate used in the invention herein is a bacteria adsorbing composition in water-insoluble form which includes a first component comprising one or more liquid permeable layers of a powerfully hydrophobic, bacteria adsorbing, physiologically innocuous material comprising a woven or non-woven hydrophilic fabric, which has been rendered hydrophobic by chemical treatment with a compound containing hydrophobic groups.

Other objects and features of the inventions will be more fully apparent from the following examples and appended claims.

### Example 1

### Manufacture of Microbe Binding Substrate

In this example the substrate of the invention is described in the following manner:

### Materials:

A. The hydrophobic substrate is preferably produced according to U.S. Pat. No. 4,617,326 and U.S. Pat. No. 7,576,256 by applying to a cellulose acetate or cotton fabric an amount of dioctadecyl carbamoyl chloride DACC or AKD as disclosed in this patent making a covalent bond between the materials. The acetate fabric is on rolls of 50 m length and at a width of 1 m. A second hydrophilic layer may be additionally added to the hydrophobic layer to absorb and hold fluids. The absorbent layer may encourage the passage of fluids through the microbe binding substrate thereby enhancing the microbe binding effect.

### Example 2

### Use of The Microbe Substrate to Bind Pathogens in a Liquid Drench

Material: Bacterial strains: 510,

Substrate as described in Example 1 Staphylococcus aureus Newman, Pseudomonas aeruginosa Enterococcus faecalis, Candida albicans Isolates were cultured on agar with 5% horse erythrocytes in 5% CO2 atmosphere at 37° C. Suspensions were made in phosphate-buffered saline (PBS, 0.02 M sodium phosphate and 0.15 M sodium chloride, pH 7.2) at 109 bacterial cells/ml, 107 fungal cells/ml or indicated concentration. The substrate was cut in 1 cm2 pieces. Incubation was made in 24 well polymer plates. 1 ml of suspension was added to each substrate piece. The plates were placed on a rotary shaker at very low speed. Incubation was performed at room temperature for the indicated time. After incubation, substrates were rinsed in PBS several times, and then put in 2.5% TCA (tricarboxylic acid).

The ATP content was measured in a luminometer (LKB Wallac). Controls: Number of adhered bacteria (CFU/ATP) were normalized against total added bacteria (CFU/ATP), and blank (no bacteria, only EDTA-Tris buffer) was the ATP value control

Results:

S. aureus>105 cells adhered during 30 sec, 1, 5 and 10 minutes, and then increased to 106 cells after 2 hrs. Some multiplication occurred during the following 24 hrs to reach 5×106 cells/cm2.

P. aeruginosa Around 106 cells adhered during 30 s,1, 5 and 10 min, and then increased during 30 and 60 min incubation to reach 107 cells/cm2 after 2 hrs incubation. No multiplication of adhered bacteria occurred during the following 24 hrs. The maximal adsorption was when 5×109 cells of S aureus were added, 108 cells adhered, for P. aeruginosa 108 cells adhered out of 109.5 added, and for E. faecalis 8×106 out of 5×1010 added. For C. albicans the slope levels off, 105 cells adhered out of 107.5 added.

Conclusion: The test substrate with the hydrophobic layer is under ideal conditions such as this liquid drench a good adsorber of different important and potential pathogens in bodily fluids.

### Example 3

### Test of the Substrate on a Rough Surface Without Applying Vacuum

A standardized pig wound model is used (BMC Surg. 2008; 8:5. Hirsch et al; Enhanced susceptibility to infections in a diabetic wound healing model) and the experimental protocol of Example 2.The maximal adsorption to the Sorbact is measured after 2 hours. When 109 cells of S aureus are added, 106 cells adhere, for P. aeruginosa 105 cells adhere out of 109.5, and for E. faecalis 1×105 out of 5×1010. For C. albicans, 103 cells adhere out of 107 added.

### Example 4

### Test of the Substrate and Vacuum on a Rough Surface

The same experimental set up as in example 3 is used but now combining the Sorbact gauze with vacuum as in example 4. The maximal adsorption to the Sorbact is measured after 2 hours. When 5×109 cells of S aureus are added, 108 cells adhere, for P. aeruginosa 107 cells adhere out of 109.5, and for E. faecalis 107 out of 1010. For C. albicans, 106 cells adhere out of 107 added.

While the invention has been described with reference to specific embodiments, it will be appreciated that numerous variations, modifications, and embodiments are possible, and accordingly, all such variations, modifications, and embodiments are to be regarded as being within the spirit and scope of the invention—spirit and scope being a dual action wound dressing having congruent opposing actions which perform a selective antimicrobial function while supporting beneficial bacteria by topically adding microbes or nutrients which may enhance the wound healing process.

What is claimed is:

1. A method for dressing a wound wherein a hydrophobic wound dressing designed to bind microorganisms is placed on a wound in combination with topically applied probiotics.

2. The method of claim 1 wherein the probiotic consists of one or more microorganism(s).

3. The method of claim 1 wherein the probiotic consists of one or more nutrient(s) identified to support microbial growth.

4. The method of claim 1 wherein the wound dressing is made of a hydrophilic substrate coated with a hydrophobic substance designed to bind microorganisms to to make the substrate hydrophobic.

5. The method of claim 1 wherein the wound dressing has a pore size greater than 0.005 microns.

6. The method of claim 1 wherein the wound dressing consists of at least one woven component.

7. A hydrophobic wound dressing designed to bind microorganisms in combination with topically applied microorganisms.

8. The wound dressing of claim 7 wherein the wound dressing is made of a hydrophilic substrate coated with a hydrophobic substance designed to bind microorganisms to to make the substrate hydrophobic.

9. The wound dressing of claim 7 wherein the wound dressing is made of a hydrophobic substrate.

10. The wound dressing of claim 7 wherein the topically applied microorganism(s) are contained within a suspension.

11. The wound dressing of claim 7 wherein the topically applied microorganism(s) are in dry form.

12. The wound dressing of claim 7 wherein the wound dressing is in physical contact with topically applied microorganism(s).

13. The wound dressing of claim 7 wherein the pore size is greater than 0.005 microns.

14. A hydrophobic wound dressing designed to bind microorganisms in combination with topically applied nutrient(s) identified to support microbial growth.

15. The wound dressing of claim 14 wherein the wound dressing is made of a hydrophilic substrate coated with a hydrophobic substance designed to bind microorganisms to make the substrate hydrophobic.

16. The wound dressing of claim 14 wherein the wound dressing is made of a hydrophobic substrate.

17. The wound dressing of claim 14 wherein the topically applied nutrient(s) identified to support microbial growth are contained within a suspension.

18. The wound dressing of claim 14 wherein the topically applied nutrient(s) identified to support microbial growth are in dry form are in dry form.

19. The wound dressing of claim 14 wherein the wound dressing is in physical contact with topically applied nutrient(s) identified to support microbial growth

20. The wound dressing of claim 14 wherein the pore size is greater than 0.005 microns.
